(19) United States
(12) Patent Application Publication
(10) Pub. No.: US 2017/0055973 A1
(43) Pub. Date: Mar. 2, 2017

(54) WOUND RETRACTORS WITH NON-CIRCULAR, NON-COPLANAR OR NON-PARALLEL INNER RINGS

(71) Applicant: Applied Medical Resources Corporation, Rancho Santa Margarita, CA (US)

(72) Inventors: Jeremy J. Albrecht, Rancho Santa Margarita, CA (US); Matthew M. Becerra, Lake Forest, CA (US); Eric Nguyen, Rancho Santa Margarita, CA (US)

(21) Appl. No.: 15/249,080

(22) Filed: Aug. 26, 2016

Related U.S. Application Data
(60) Provisional application No. 62/210,576, filed on Aug. 27, 2015.

(51) Int. Cl. A61B 17/02

(52) U.S. Cl. CPC A61B 17/0218; A61B 17/0293; A61B 2017/0225; A61B 2017/3429

(57) ABSTRACT

A retractor/protector suitable for use in a surgical incision or a natural orifice comprises a longitudinal axis defining an instrument access channel extending from a proximal end to a distal end; an outer ring; an inner ring; and a flexible sheath extending between the outer ring and the inner ring, wherein the instrument access channel extends through the outer ring, the inner ring, and the sheath, and the inner ring is non-circular, non-coplanar and/or non-parallel. Embodiments of the retractor/protector are described that have inner rings of a variety of non-circular or non-coplanar shapes that are insertable through small incisions or orifices, minimize space occupied and maintain a strong anchor and effective retraction.

### CROSS REFERENCE TO RELATED APPLICATIONS

This application claims the benefit of U.S. Application No. 62/210,576, filed Aug. 27, 2015, the entire disclosure of which is incorporated by reference.

### TECHNICAL FIELD

This invention generally relates to medical devices and, more specifically, to a wound retractor/protector having non-circular, non-coplanar and/or non-parallel inner rings.

### DISCUSSION OF THE RELEVANT ART

Wound retractors/protectors have been described in U.S. Pat. Nos. 7,650,887; 7,727,146; 7,883,461; 7,913,697; 8,235,054, and 8,267,858, the disclosures of which are hereby incorporated by reference in their entireties. The basic components of such wound retractors include an outer ring or rings, which may be flexible or rigid, an inner ring, and flexible sleeve attached at either end to the outer and inner ring.

In use, the inner ring of the wound retractor is compressed for insertion through an incision. Once placed, the inner ring requires a particular “bounce back” force to ensure that while the device is retracted and in use during surgery, the inner ring does not collapse, which would result in loss of retraction.

Working space inside the body cavity is always limited, especially when using surgical instruments and devices. Typically, a wound retractor's inner ring is designed with a circular shape and a solid cross-sectional profile, a configuration that provides a solid engagement of the inner ring with the inner body cavity wall. However, such rings occupy valuable space with the body cavity and may be difficult to deploy through small incisions or into small natural orifices. What is needed, therefore, is an inner ring design that is easy to deploy and maximizes inner working spaces, while maintaining effective retracting characteristics.

### SUMMARY

A retractor/protector suitable for use in a surgical incision or a natural orifice comprises a longitudinal axis defining an instrument access channel extending from a proximal end to a distal end; an outer ring; an inner ring; and a flexible sheath extending between the outer ring and the inner ring, wherein the instrument access channel extends through the outer ring, the inner ring, and the sheath, and the inner ring is non-circular. In some embodiments, the inner ring is elliptical. In other embodiments, the inner ring is generally semicircular. In some embodiments, the inner ring comprises a pair of spokes or at least two pairs of spokes. In other embodiments, the inner ring comprises a plurality of spokes. In some embodiments, the outer ring is also non-circular.

A retractor/protector suitable for use in a surgical incision or a natural orifice comprises a longitudinal axis defining an instrument access channel extending from a proximal end to a distal end; an outer ring; an inner ring; and a flexible sheath extending between the outer ring and the inner ring, wherein the instrument access channel extends through the outer ring, the inner ring, and the sheath, and the inner ring is non-coplanar. In some embodiments, the inner ring comprises a first portion, a second portion, and a pair of step portions, the first portion and second portion being connected to each other by the pair of step portions. In another embodiment, the inner ring comprises a curved first portion, an opposed curved second portion, a rectangular first portion, an opposed rectangular second portion, and two pairs of step portions, each curved portion being connected on either end to a rectangular portion by a pair of step portions.

A retractor/protector suitable for use in a surgical incision or a natural orifice comprises a longitudinal axis defining an instrument access channel extending from a proximal end to a distal end; an outer ring; an inner ring; and a flexible sheath extending between the outer ring and the inner ring, wherein the instrument access channel extends through the outer ring, the inner ring, and the sheath, and the inner ring is non-parallel to the outer ring. In some embodiments, the inner ring is attached to the sheath at a ninety degree angle to the outer ring.

### BRIEF DESCRIPTION OF THE DRAWINGS

FIG. 1 illustrates an isometric view of an adjustable wound retractor/protector.

FIG. 2 is a cross-sectional view of a wound retractor/protector deployed in an incision.

FIG. 3A illustrates a top view of a wound retractor/protector having an elliptical inner ring.

FIG. 3B illustrates a front view of a wound retractor/protector having an elliptical inner ring.

FIG. 3C illustrates a bottom view of a wound retractor/protector having an elliptical inner ring.

FIG. 3D illustrates a right view of a wound retractor/protector having an elliptical inner ring.

FIG. 3E illustrates an auxiliary view of a wound retractor/protector having an elliptical inner ring.

FIG. 4A illustrates a top view of a wound retractor/protector having an inner ring with spokes.

FIG. 4B illustrates a front view of a wound retractor/protector having an inner ring with spokes.

FIG. 4C illustrates a bottom view of a wound retractor/protector having an inner ring with spokes.

FIG. 4D illustrates a right view of a wound retractor/protector having an inner ring with spokes.

FIG. 4E illustrates an auxiliary view of a wound retractor/protector having an inner ring with spokes.

FIG. 5A illustrates a top view of a wound retractor/protector having a semi-circular inner ring.

FIG. 5B illustrates a front view of a wound retractor/protector having a semi-circular inner ring.

FIG. 5C illustrates a bottom view of a wound retractor/protector having a semi-circular inner ring.

FIG. 5D illustrates a right view of a wound retractor/protector having a semi-circular inner ring.

FIG. 5E illustrates an auxiliary view of a wound retractor/protector having a semi-circular inner ring.

FIG. 6A illustrates a top view of a wound retractor/protector having an elliptical inner ring with spokes.

FIG. 6B illustrates a front view of a wound retractor/protector having an elliptical inner ring with spokes.

FIG. 6C illustrates a bottom view of a wound retractor/protector having an elliptical inner ring with spokes.

FIG. 6D illustrates a right view of a wound retractor/protector having an elliptical inner ring with spokes.

FIG. 6E illustrates an auxiliary view of a wound retractor/protector having an elliptical inner ring with spokes.

FIG. 7A illustrates a top view of an alternative embodiment of a wound retractor/protector having an elliptical inner ring with spokes.

FIG. 7B illustrates a front view of an alternative embodiment of a wound retractor/protector having an elliptical inner ring with spokes.

FIG. 7C illustrates a bottom view of an alternative embodiment of a wound retractor/protector having an elliptical inner ring with spokes.

FIG. 7D illustrates an auxiliary view of an alternative embodiment of a wound retractor/protector having an elliptical inner ring with spokes.

FIG. 8A illustrates a top view of a wound retractor/protector having a noncircular inner ring and a noncircular outer ring.

FIG. 8B illustrates a right view of a wound retractor/protector having a noncircular inner ring and a noncircular outer ring.

FIG. 8C illustrates an auxiliary view of a wound retractor/protector having a noncircular inner ring and a noncircular outer ring.

FIG. 9A illustrates a top view of a wound retractor/protector having a non-coplanar inner ring.

FIG. 9B illustrates a front view of a wound retractor/protector having a non-coplanar inner ring.

FIG. 9C illustrates a bottom view of a wound retractor/protector having a non-coplanar inner ring.

FIG. 9D illustrates an auxiliary view of a wound retractor/protector having a non-coplanar inner ring.

FIG. 10A illustrates a top view of the inner ring of FIGS. 9A-D.

FIG. 10B illustrates a front view of the inner ring of FIGS. 9A-D.

FIG. 10C illustrates an auxiliary view of the inner ring of FIGS. 9A-D.

FIG. 11A illustrates a top view of an alternative embodiment of a wound retractor/protector having a non-coplanar inner ring.

FIG. 11B illustrates a front view of an alternative embodiment of a wound retractor/protector having a non-coplanar inner ring.

FIG. 11C illustrates a side view of an alternative embodiment of a wound retractor/protector having a non-coplanar inner ring.

FIG. 11D illustrates an auxiliary view of an alternative embodiment of a wound retractor/protector having a non-coplanar inner ring.

FIG. 12A illustrates a top view of the inner ring of FIGS. 11A-D.

FIG. 12B illustrates a front view of the inner ring of FIGS. 11A-D.

FIG. 12C illustrates a bottom view of the inner ring of FIGS. 11A-D.

FIG. 13A illustrates a top view of an alternative embodiment of a wound retractor/protector having a non-parallel inner ring.

FIG. 13B illustrates a front view of an alternative embodiment of a wound retractor/protector having a non-parallel inner ring.

FIG. 13C illustrates a bottom view of an alternative embodiment of a wound retractor/protector having a non-parallel inner ring.

FIG. 13D illustrates an auxiliary view of an alternative embodiment of a wound retractor/protector having a non-parallel inner ring.

### DETAILED DESCRIPTION

Wound retractors/protectors have been described in U.S. Pat. Nos. 7,650,887; 7,727,146; 7,883,461; 7,913,697; 8,235,054, and 8,267,858, and U.S. application Ser. No. 12/873,115, the disclosures of which are hereby incorporated by reference in their entireties.

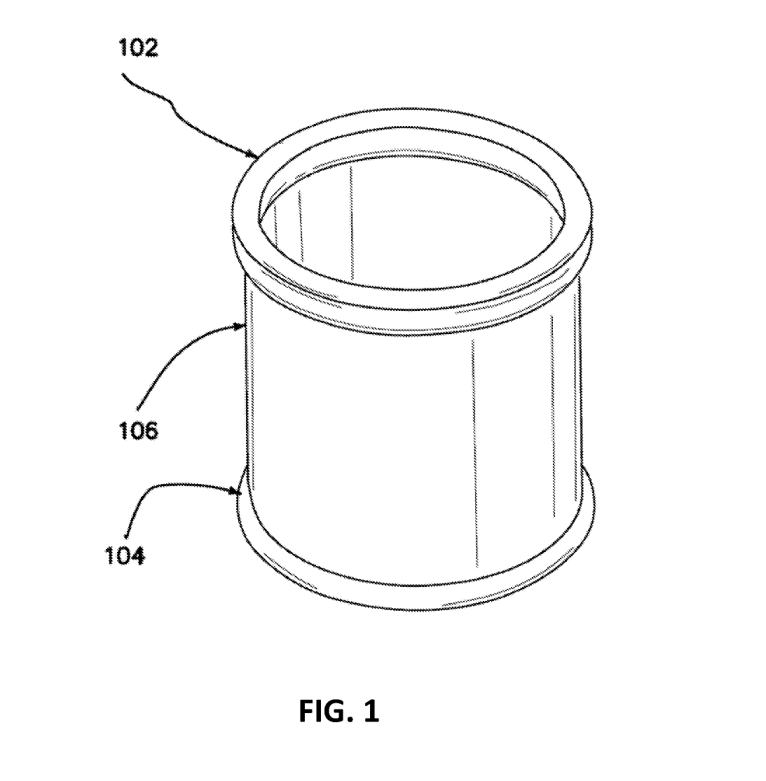

FIG. 1 illustrates an adjustable wound retractor useful in a variety of surgical procedures. The wound retractor includes an outer ring 102, an inner ring 104, and a distensible sleeve 106 coupling the outer ring and the inner ring. Outer ring 102 is shown as a single ring, but may also be a double ring or triple ring or multiples thereof.

In the illustrated embodiment, the outer ring 102 comprises an annular axis around which the outer ring 102 is rotatable or invertible in a process through which the outer ring 102 is rolled through itself. Consequently, the outer ring 102 comprises a flexible material. In some embodiments, the flexible material comprises one or more polymers, for example, flexible engineering plastics. In some embodiments, the flexible material comprises an elastomer, for example, a thermoplastic elastomer. In some embodiments, the outer ring 102 comprises a composite, for example, a polymer and a reinforcing material. Examples of suitable reinforcing materials include fibers, fabrics, and the like, which comprise at least one of polymer, metal, glass, ceramic, and the like. Embodiments of the outer ring 102 are molded and/or extruded as a single piece or as a plurality of pieces that are assembled into the outer ring 102.

In the illustrated embodiment, a cross-sectional shape of the outer ring 102 is generally a figure-8, or first circle and a second circle joined by a web extending therebetween. The outer ring may be solid or may have one or more lumens disposed in the ring. Other embodiments of the outer ring have different cross-sectional shapes, for example, generally oval or elliptical; diamond-shaped or rhomboid; hourglass or dog bone shaped; snowman-shaped; radially flat (washer-shaped outer ring), longitudinally flat (cylindrical outer ring), or flat at another angle (frustoconical outer ring); circular (toroidal outer ring), X-shaped, triangular, square, hexagonal, polygonal, and the like. Some embodiments of the outer ring comprise one or more gripping surfaces that facilitate manually rolling the outer ring around the annular axis thereof. Examples of suitable gripping surfaces include generally flattened surfaces and concave surfaces. Some embodiments of the outer ring 102 have a Möbius configuration in which the outer ring 102 is fabricated with a preloaded circumferential torsional stress, for example, by twisting an elongate member followed by joining the ends.

In some hollow embodiments of the outer ring 102, a wire or rod is disposed in at least one first lumen. Some embodiments of the outer ring 102 do not comprise a rod or wire disposed in a lumen thereof. Some embodiments of a non-compliant outer ring 102 facilitate direct coupling of another device to the outer ring 102 for example, a lid, cap, and/or gel cap. Some embodiments of a compliant outer ring 102 conform to a body surface.

Returning to FIG. 1, the sleeve 106 may be coupled to the outer ring 102 and the inner ring 104 by heat seal, adhesive, or other means that are well known in the art. The sleeve 106 may be made of a material that is flexible and impermeable to fluids and bacteria.

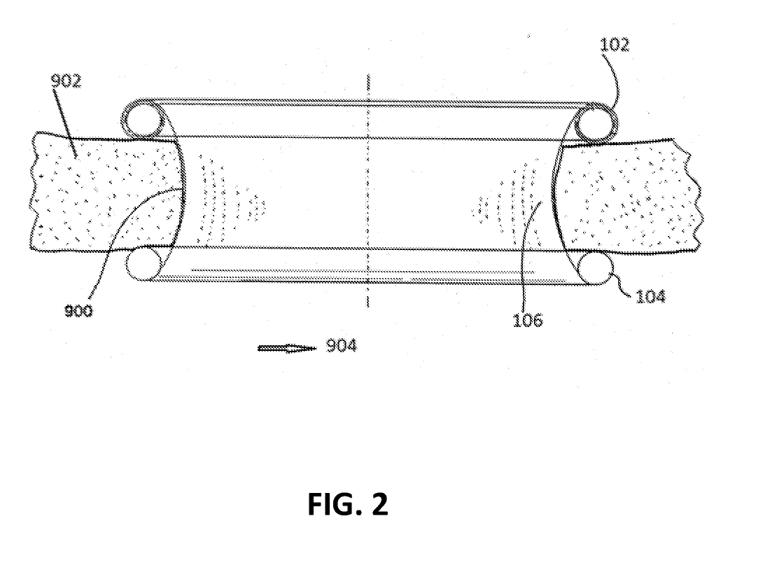

The inner ring 104 may be made of materials of sufficient hardness to retain its shape after insertion into a body cavity 904 (FIG. 2) but sufficiently flexible so as to allow the inner ring to be compressed for insertion through an incision. The materials of which the outer ring 102 is made must allow the outer ring to be turned around its annular axis as described above. The shape of the outer ring 102 affects both its ability to grip and to provide stability during and after adjustment.

FIG. 2 illustrates the wound retractor deployed in a wound opening 900. To deploy the wound retractor, an incision in the shape of a slit is first made in the body wall 902 of a patient, such as the abdominal wall 902. The inner ring 104 is compressed and the inner ring and sleeve 106 are manually inserted into the body cavity 904 through the incision with the outer ring 102 remaining external to the body cavity. Once the inner ring 104 is within the body cavity 904, it expands around the inner surface of the incision 900 so as to be generally parallel to the outer surface of the abdominal wall 902. The sleeve 106 provides a working channel from outside the body cavity 904 to inside the body cavity.

The outer ring 102 initially rests above the abdominal wall 902 around the wound opening 900. Since the upper end of the sleeve 106 is coupled to the outer ring 102, the sleeve 106 can be drawn upwards and radially outward or inward, thereby drawing the inner ring 104 tightly against the inner surface of the abdominal wall 902. Moreover, the intermediate portion of the sleeve 106 is drawn tightly against the sides and edges of the wound opening 900, thereby retracting the adjacent tissue and producing a tightly sealed opening in the body cavity 904. The sleeve 106 contacts the entire surface of the wound 900 and protectively covers and seals it from contamination and infection. Depending on the size and depth of the incision 900, the user can roll up the sleeve 106 by gripping the outer ring 102 and rotating it until the sleeve 106 abuts the outer edge of the wound opening 900. The inner ring 104 is adapted for juxtaposition with the inner surface of the abdominal wall 902 and the outer ring 102 is adapted for juxtaposition with the outer surface of the abdominal wall. Both the inner ring 104 and the outer ring 102 are adapted for disposition relative to the incision 900 in the abdominal wall 902. The sleeve 106 is adapted to traverse the incision 900 in the abdominal wall 902.

After surgery, the wound retractor may be retrieved by grabbing the inner ring 104 and the sleeve 106 and pulling them through the wound opening 900. The use of the sleeve 106 and the ease of retracting the outer ring 102 provide higher compression between the inner and outer rings. As a result, the wound retractor/protector provides incremental adjustability to fit a wide range of incision sizes and isolates and protects the wound from bacterial infection as diseased body parts and contaminated instruments are passed through the wound.

The wound retractor/protector shown in FIG. 1 is useful in a wide range of surgical procedures, both when deployed through surgical incisions as described above or when used in natural bodies cavities such as the rectum or vagina. That wound retractor's inner ring is designed with a circular shape and a solid cross-sectional profile, a configuration that provides a solid engagement of the inner ring with the inner body cavity wall. However, such rings occupy valuable space with the body cavity and may be difficult to deploy, particularly where working space inside the body cavity is limited. They may also be difficult to insert through small incisions. The present invention is directed to an inner ring design that is easy to deploy through small incisions or orifices and that maximizes inner working spaces, while maintaining effective retracting characteristics. To that end, several inner ring configurations were invented that minimize space occupied while maintaining a strong anchor and effective retraction.

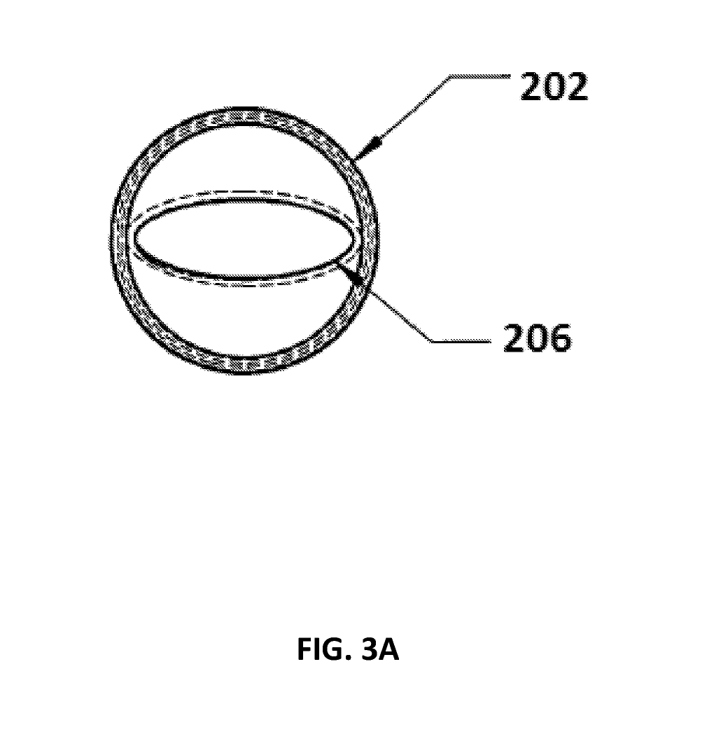

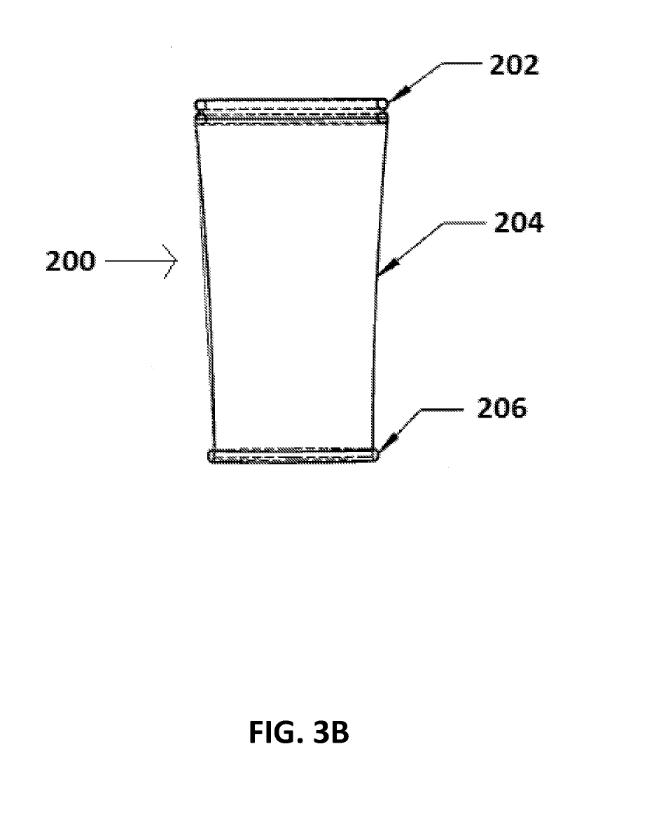

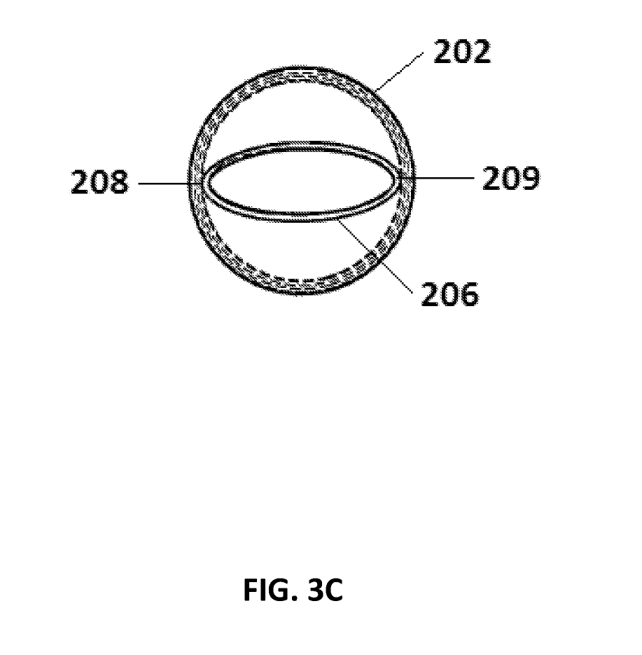

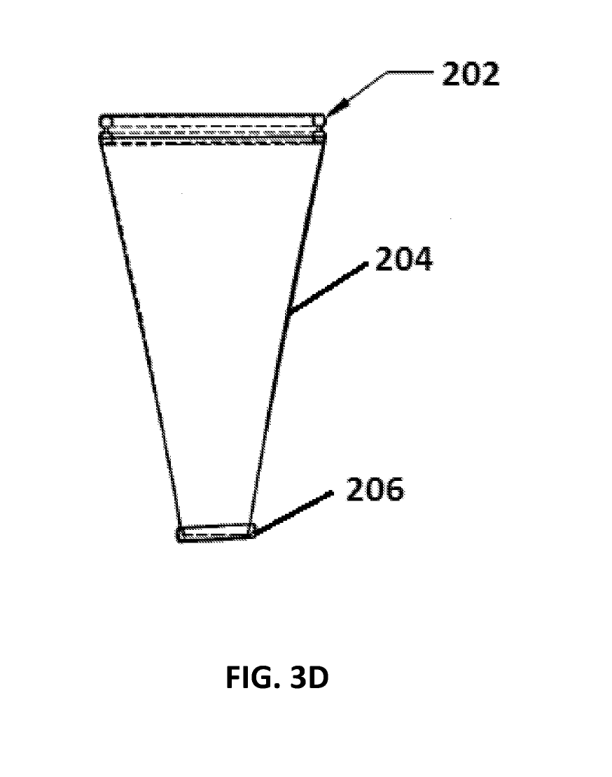

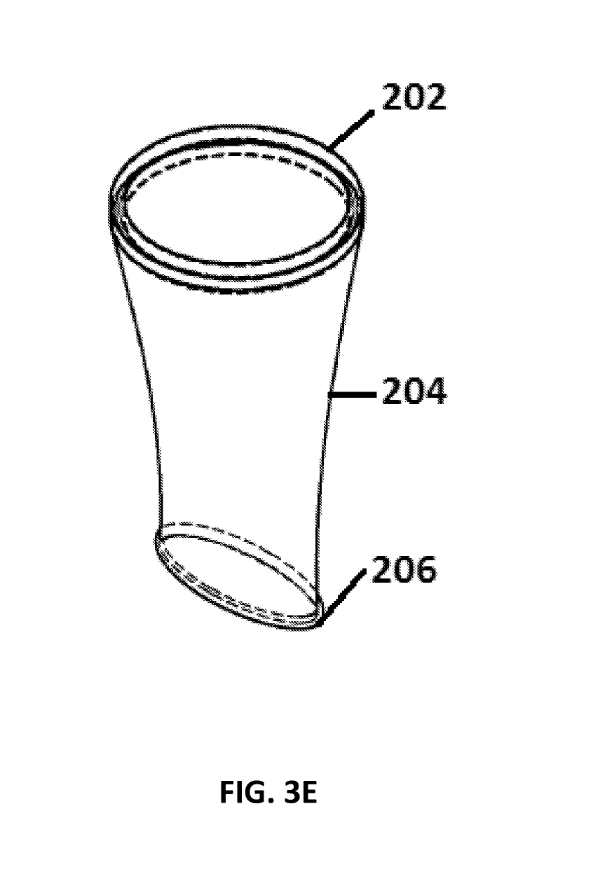

FIGS. 3A,BC illustrate a top, front and bottom view of a wound retractor/protector 200 having an outer ring 202, a sheath 204 and a non-circular inner ring 206 designed to maximize inner working spaces and ease of deployment while maintaining effective retracting characteristics. The inner ring 206 in this embodiment is elliptical, providing 2 apexes 208, 209 located at either end of the major axis of the ellipse. In front view (FIG. 3B), the retractor/protector of this embodiment resembles the retractor/protector of FIG. 1, at least where the major axis of the elliptical inner ring has the same or similar diameter as the outer ring. When viewed from the side, however, the reduced footprint of the inner ring is apparent (see FIG. 3D). An auxiliary view of the elliptical retractor/protector is shown in FIG. 3E.

In use, the elliptical inner ring is placed into the incision perpendicular to the incision, creating an unbalanced tension distribution between the circular outer ring and the elliptical inner ring. To offset this balance state, tension force is focused at the two apexes, forcing the inner ring to become more circular like the outer ring. This, in turn, forces the wound to open in the same manner as in the wound retractor that has circular inner and outer rings.

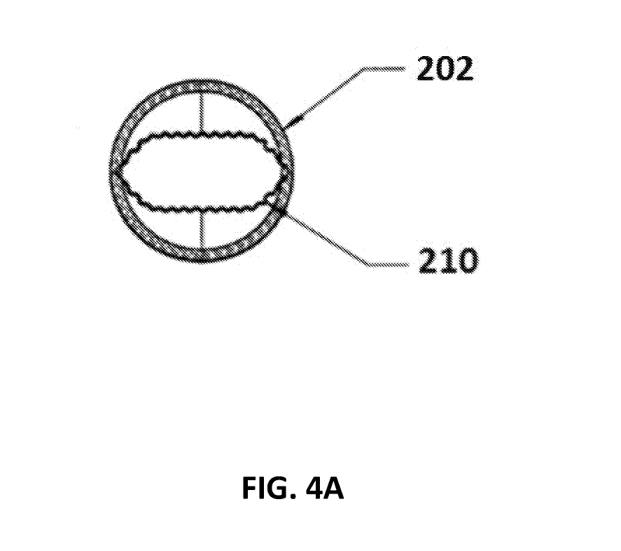

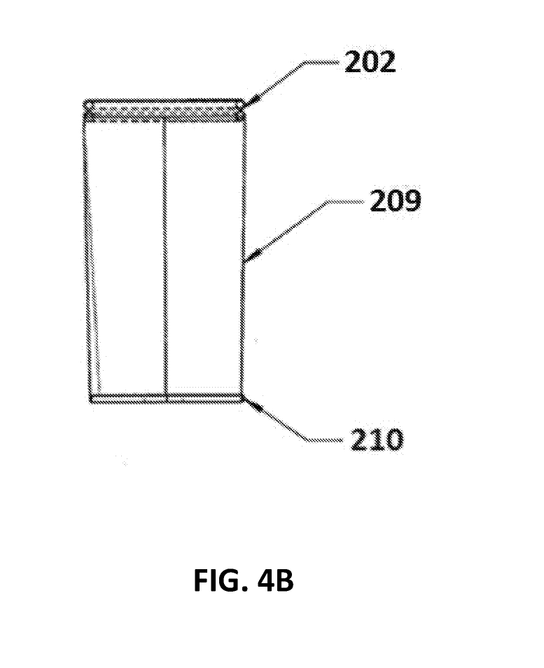

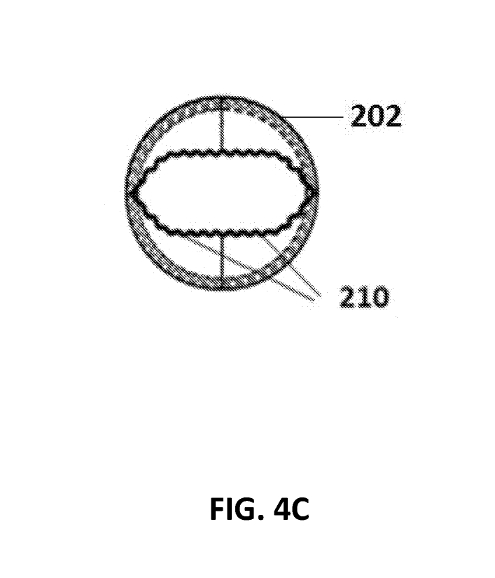

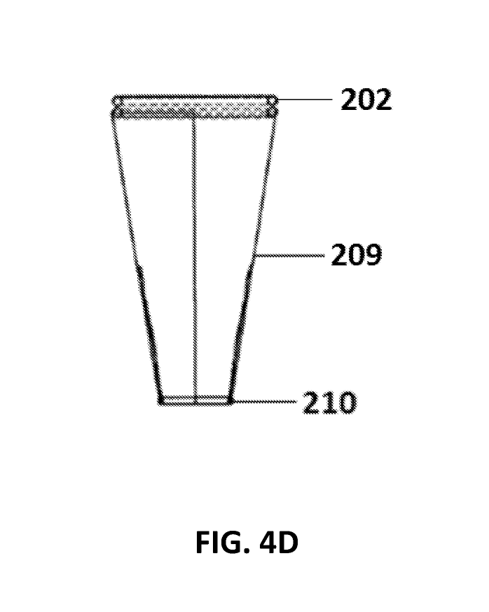

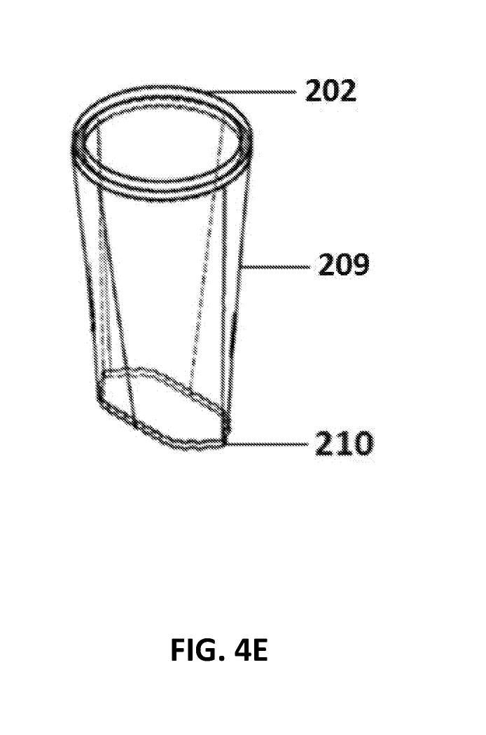

A variation of the elliptical inner ring is shown in FIG. 4A-E. In this embodiment, the wound retractor/protector has an outer ring 202, an elliptical inner ring with spokes 210 distributed around and projecting out from the ellipse (see FIGS. 4A, 4C, and 4E) and a sheath 209 connecting the two rings. As with the embodiment of FIG. 3, the front view (FIG. 4B) resembles the retractor/protector of FIG. 1, at least where the major axis of the elliptical inner ring has the same or similar diameter as the outer ring. When viewed from the side, however, the reduced footprint of the inner ring is apparent (see FIG. 4D).

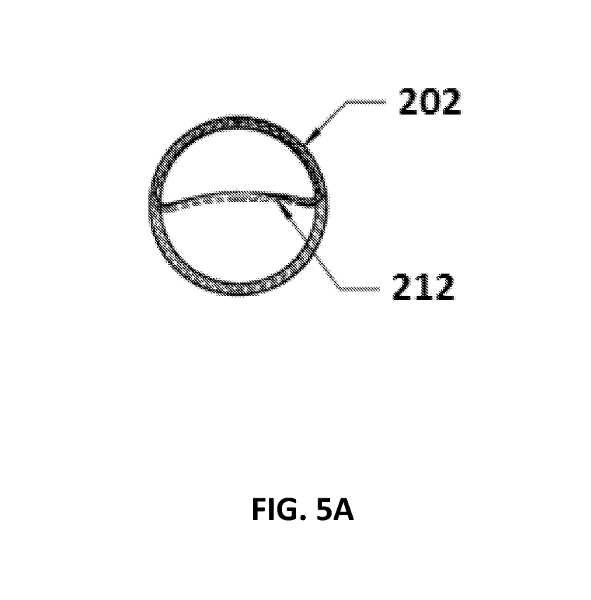

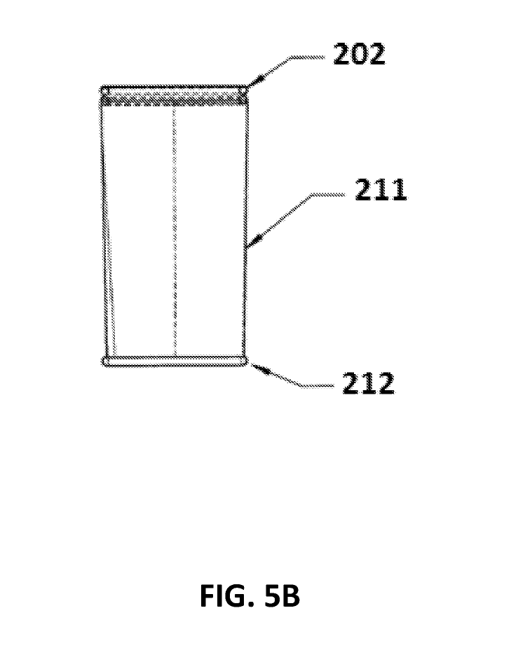

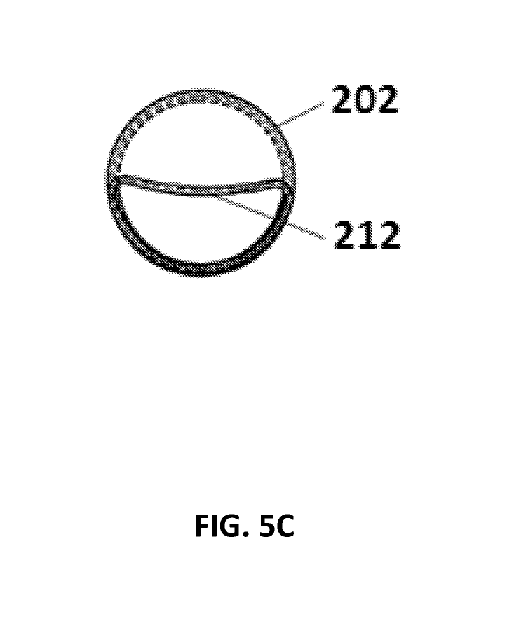

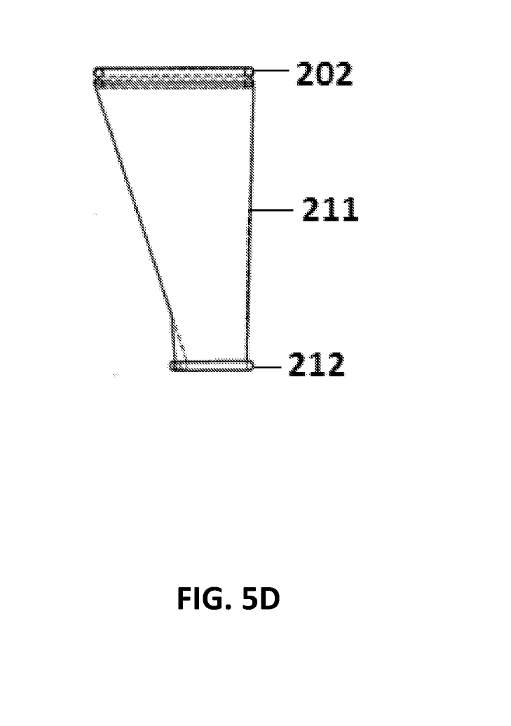

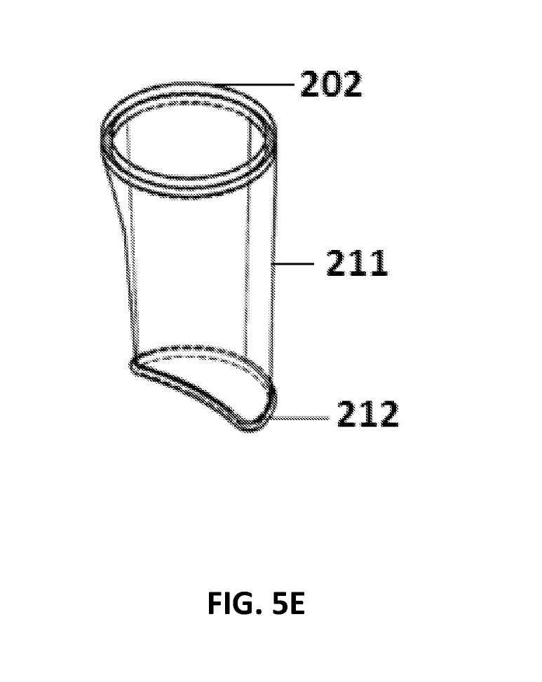

FIG. 5 shows a wound retractor/protector having an outer ring 202, a generally semicircular inner ring 212, best seen in FIGS. 5A, 5C and 5E, and a sheath 211 connecting the two rings. As with the elliptical inner ring, the semi-circular inner ring has a reduced footprint, best seen in FIGS. 5D and 5E. The diameter of the semicircular ring may be straight or curved, forming a crescent moon shape, as shown in FIG. 5.

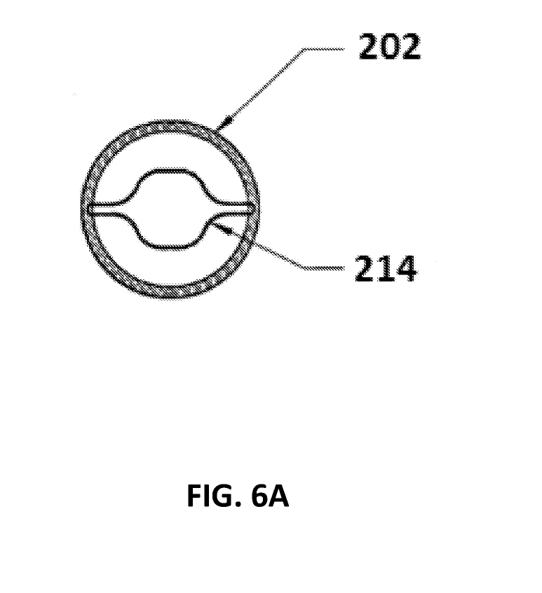

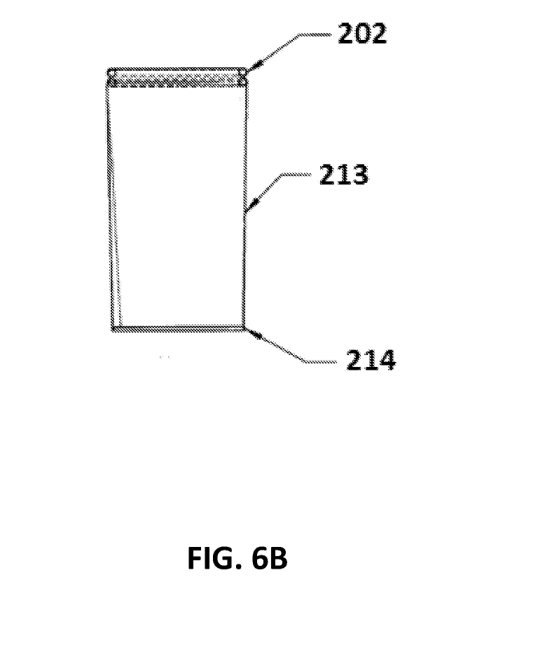

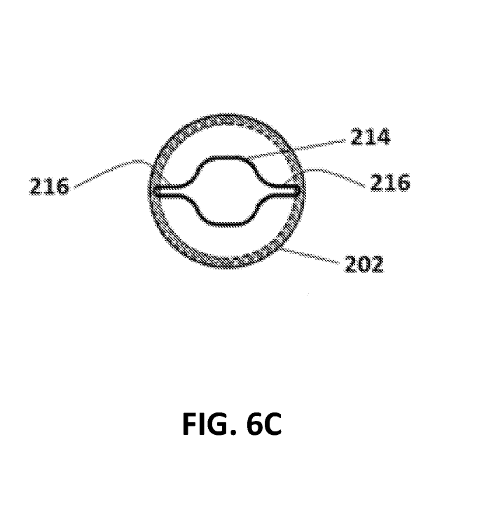

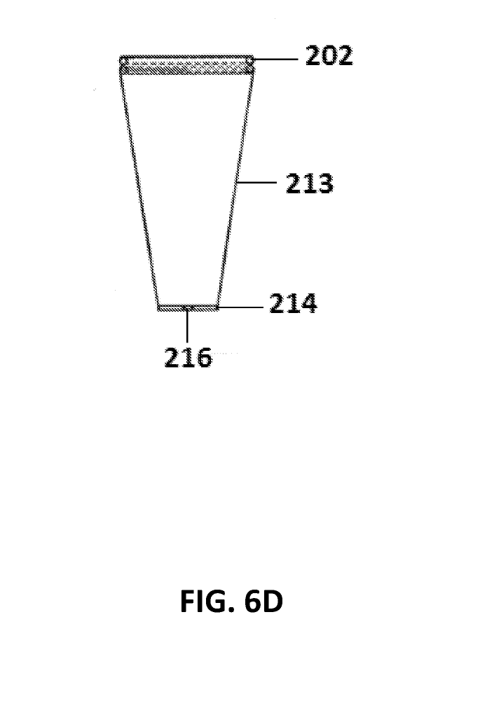

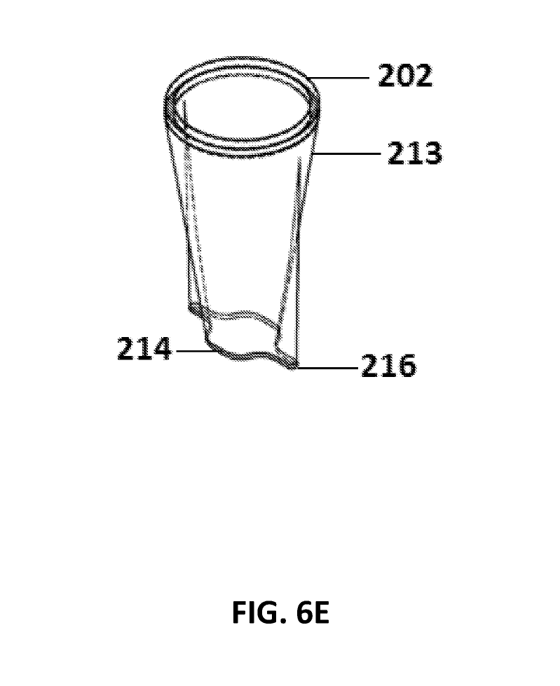

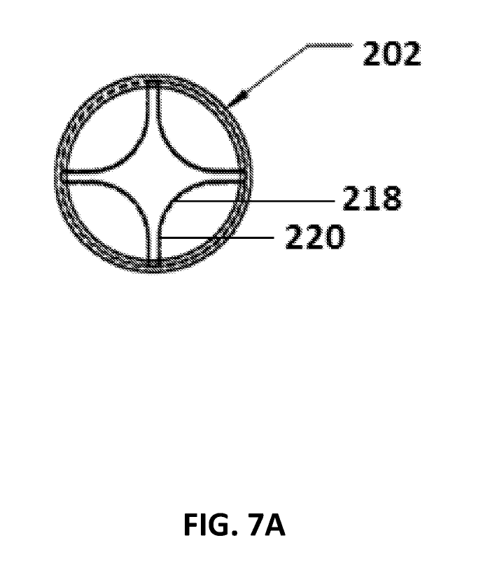

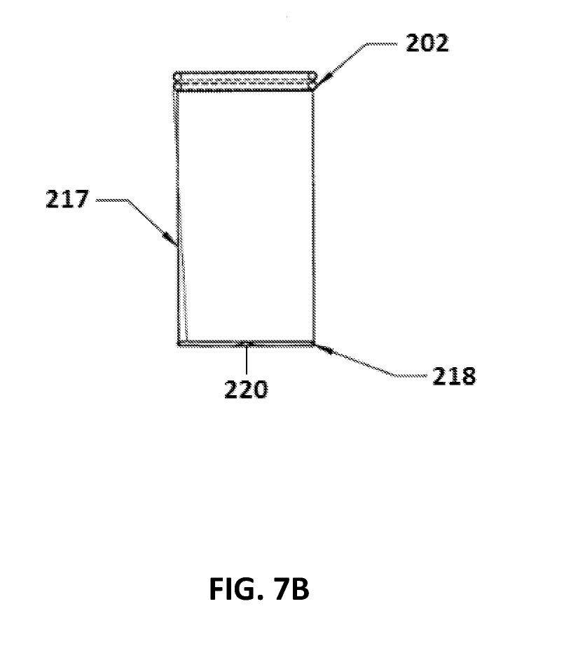

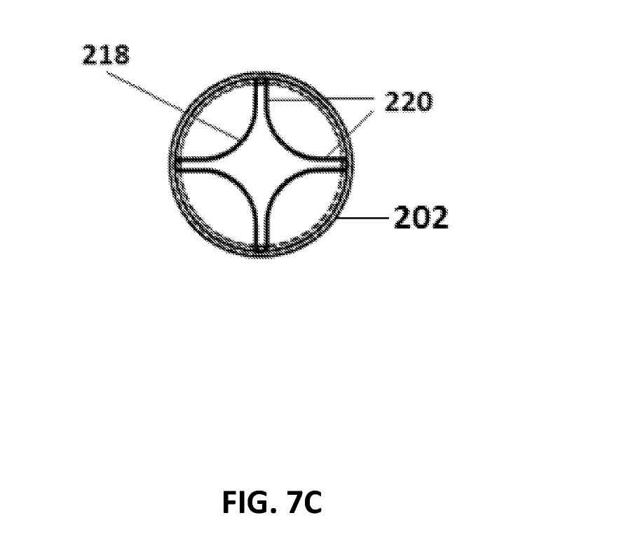

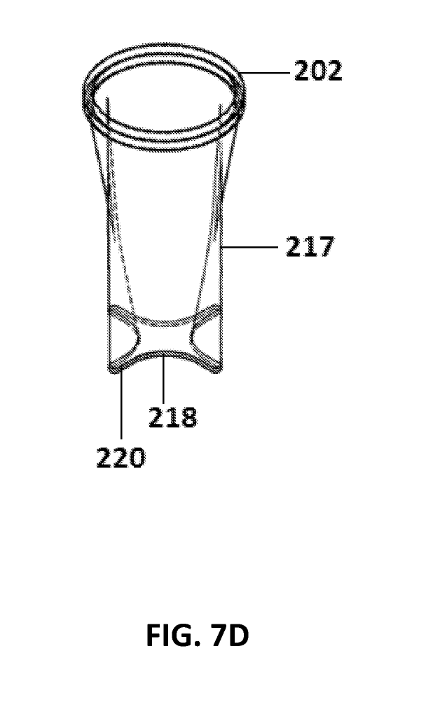

FIG. 6 shows a wound retractor having an outer ring 202, a roughly elliptical inner ring 214 with a pair of opposing spokes 216, and a sheath 213 connecting the two rings. An alternative to this embodiment is shown in FIG. 7, where the sheath 217 is connected to the outer ring 202 and an inner ring 218 with two pairs of opposing spokes 220.

These particular geometries allow the inner ring to achieve higher retention when subject to tension exerting from the outer ring during retraction. The high retention force from the inner ring's apexes allow further tensioning, or retracting, from the outer ring, thus creating larger wound openings without the need to make a larger incision.

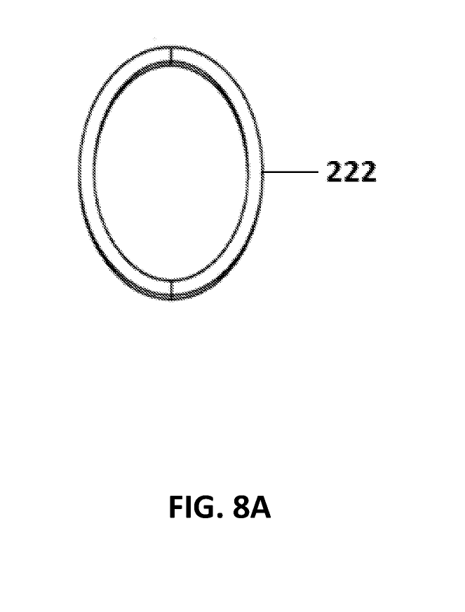

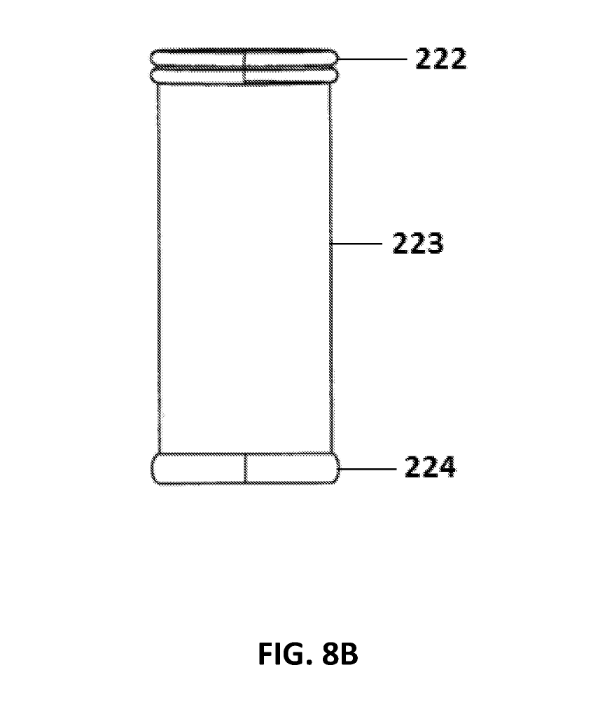

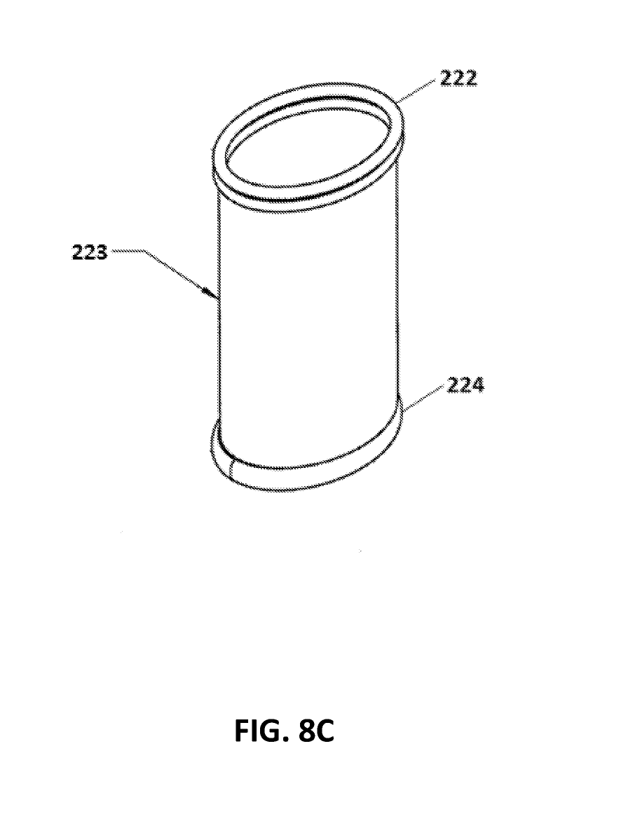

In yet another embodiment of the present invention, both the inner and the outer ring can be non-circular, which may be particularly useful where access space is limited. FIG. 8 shows a wound retractor having a non-circular outer ring 222 and a noncircular inner ring 224 connected by a sheath 223.

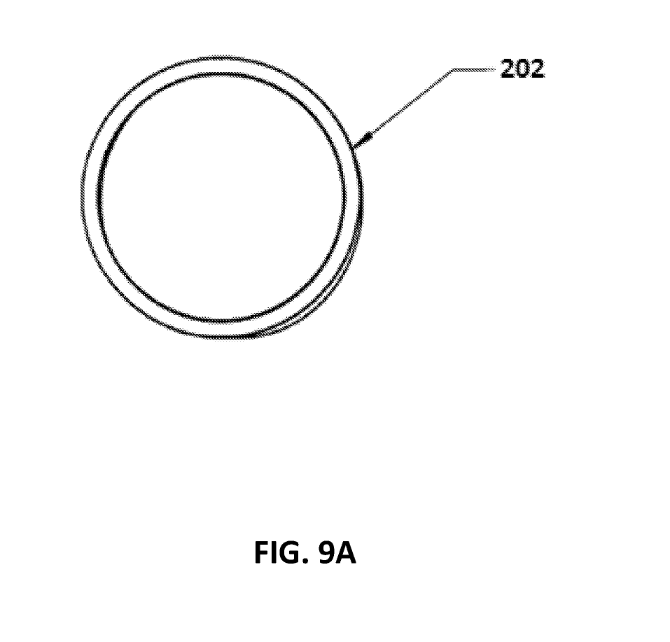

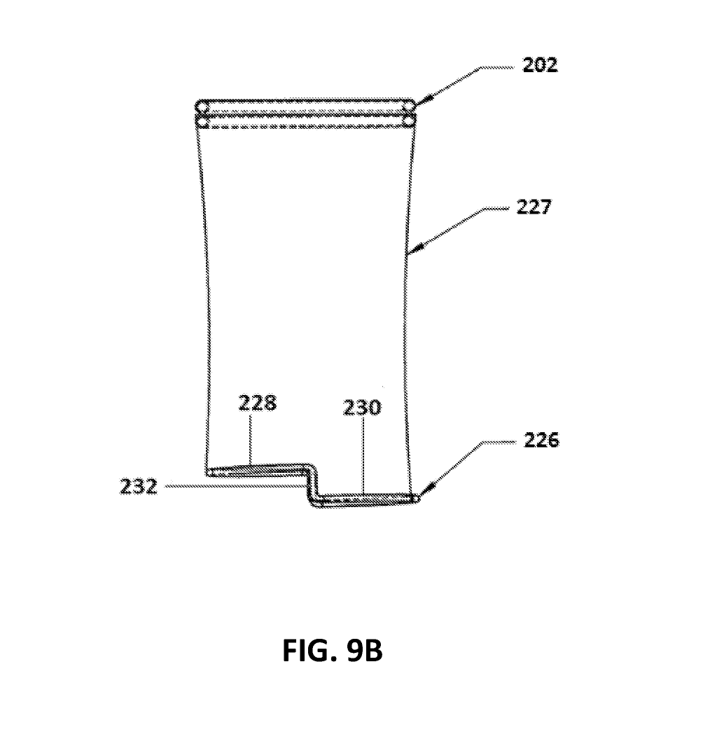

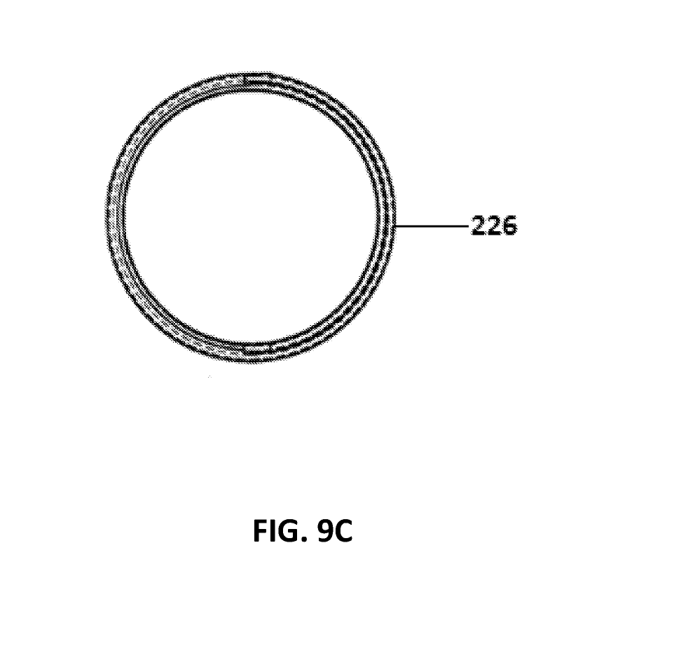

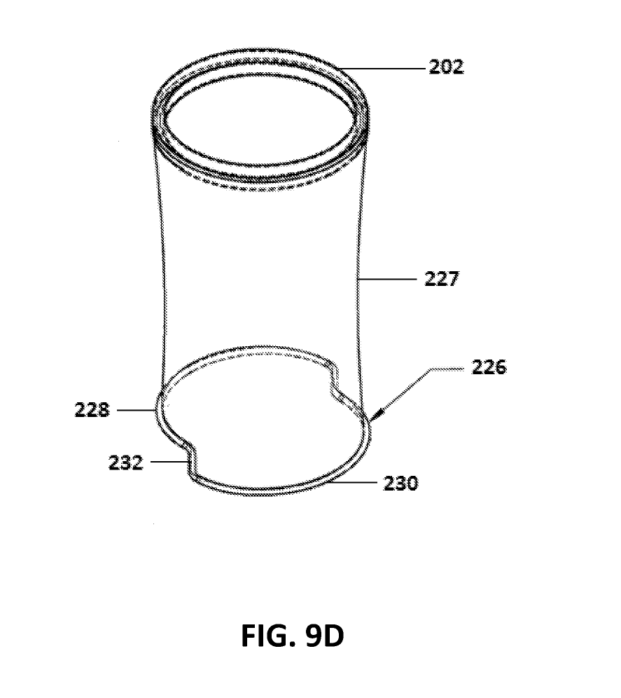

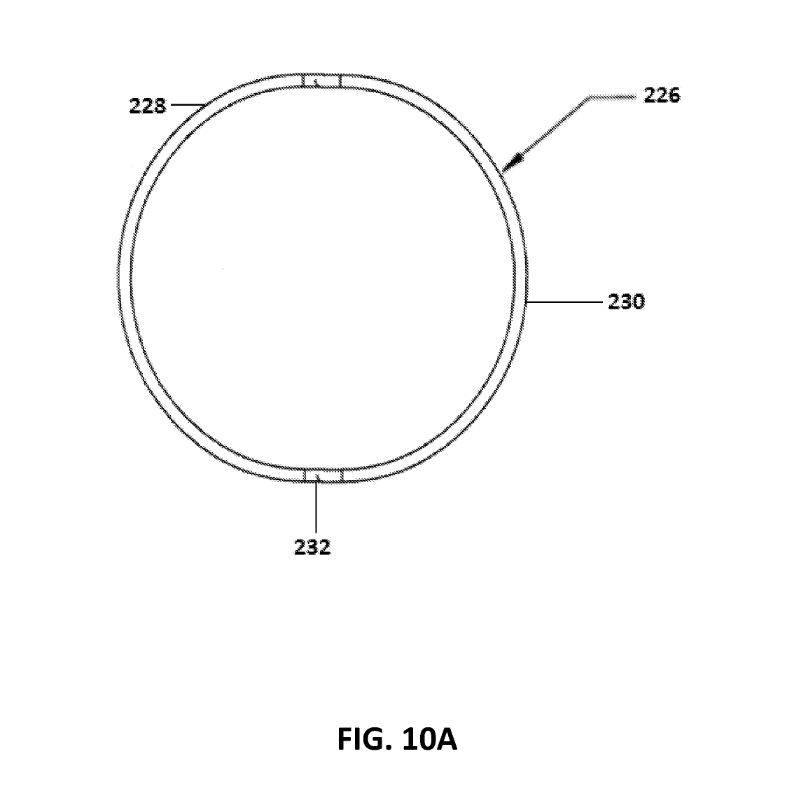

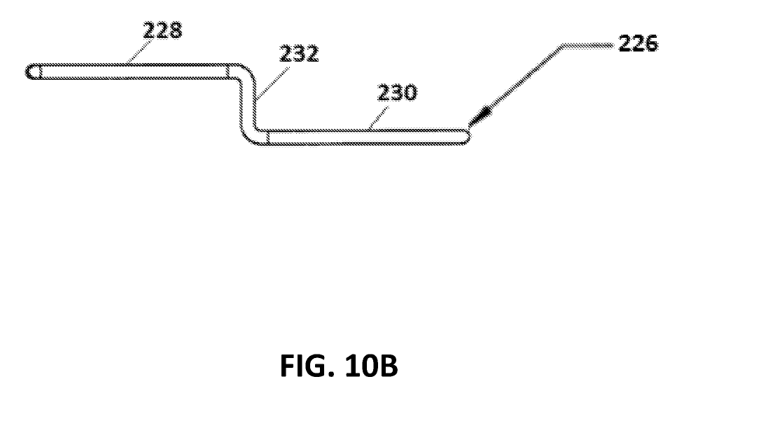

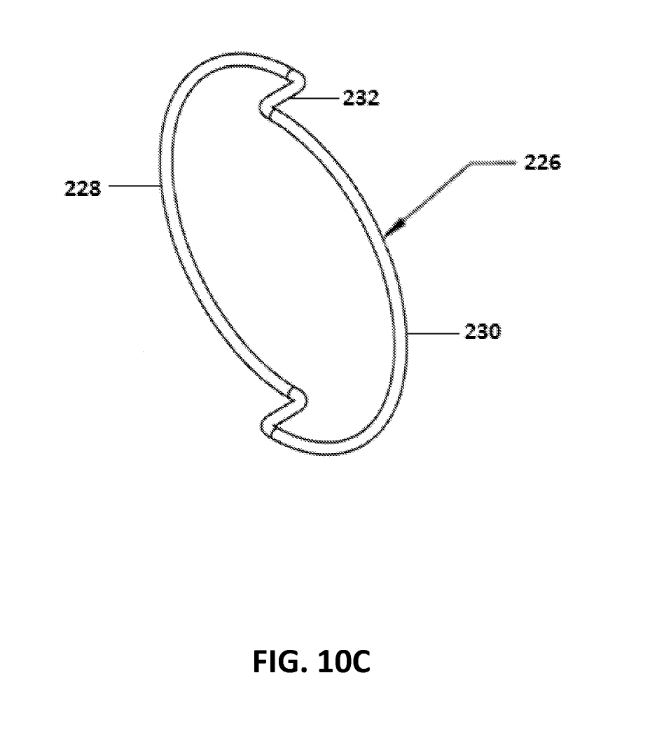

In other design alternatives, the inner ring can be configured to be a non-coplanar circular ring (see FIGS. 9-12). In FIG. 9, a circular outer ring 202 and a non-coplanar inner ring 226 are each joined to opposite ends of a sheath 227. The inner ring 226, shown more closely in FIG. 10, has a first portion 228 and a second portion 230 that are displaced from each other by a step portion 232, resulting in the non-coplanar ring.

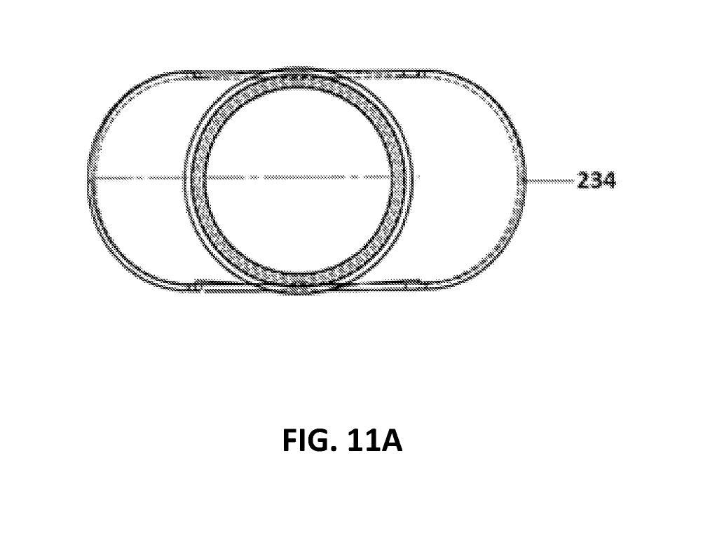

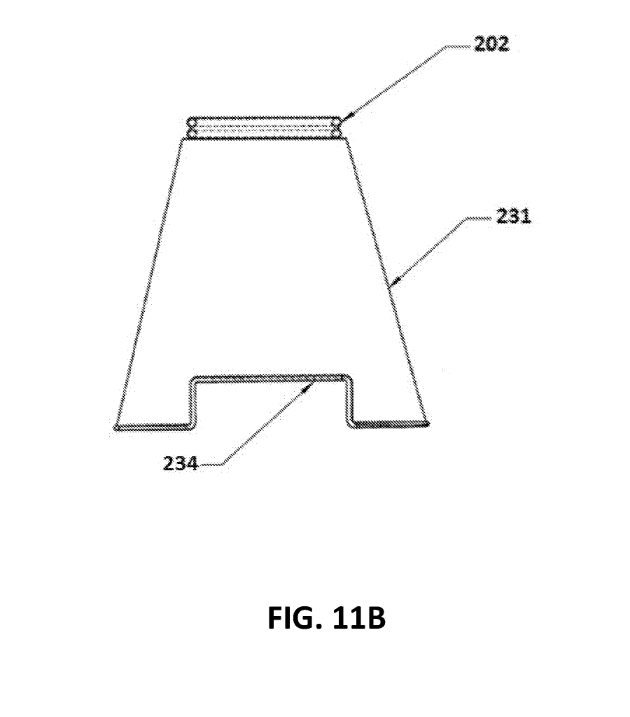

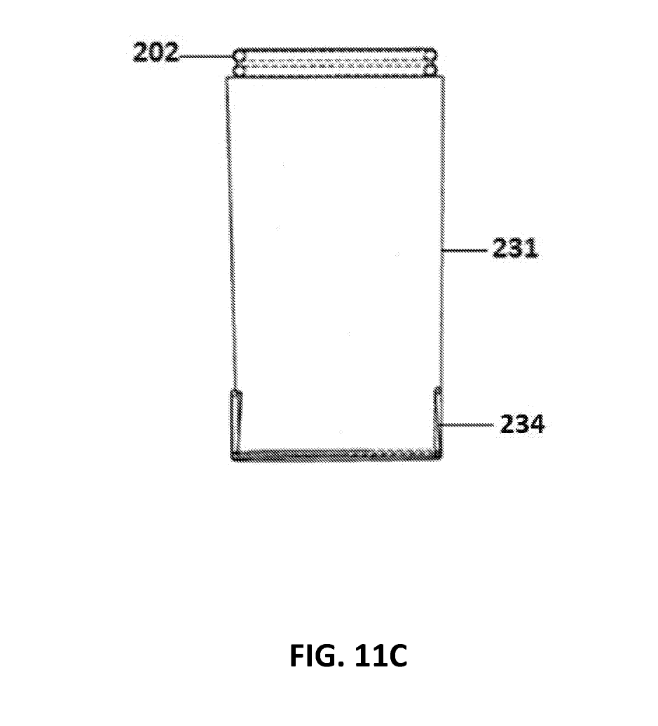

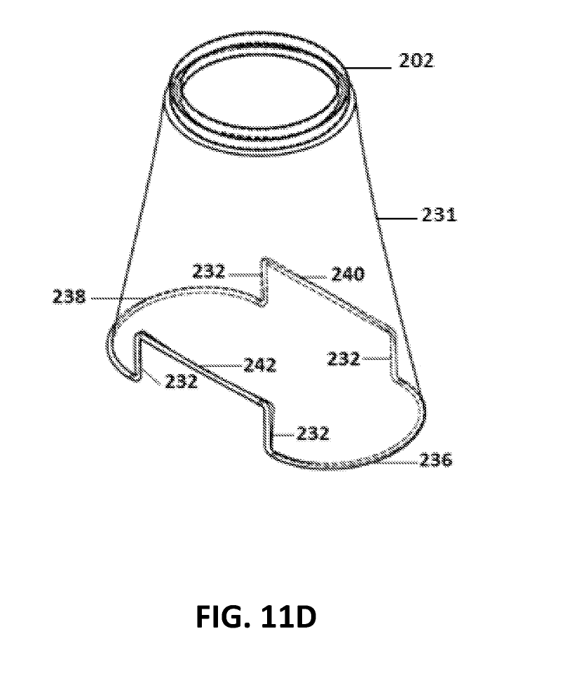

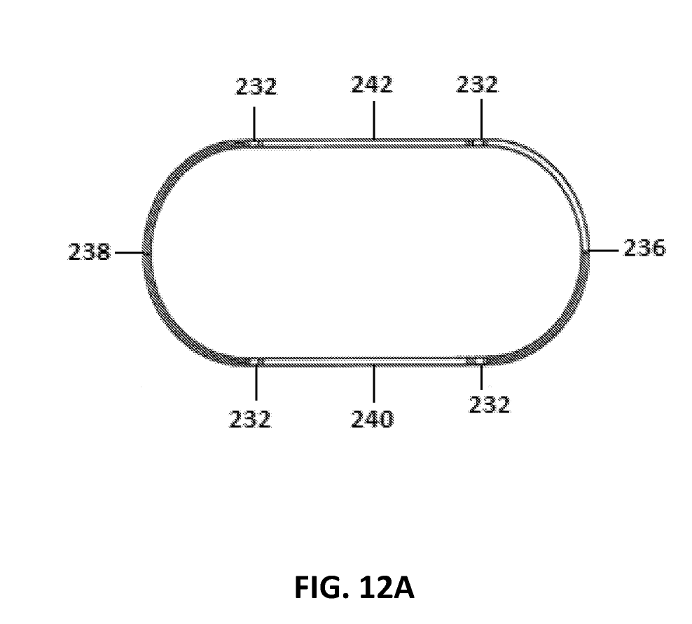

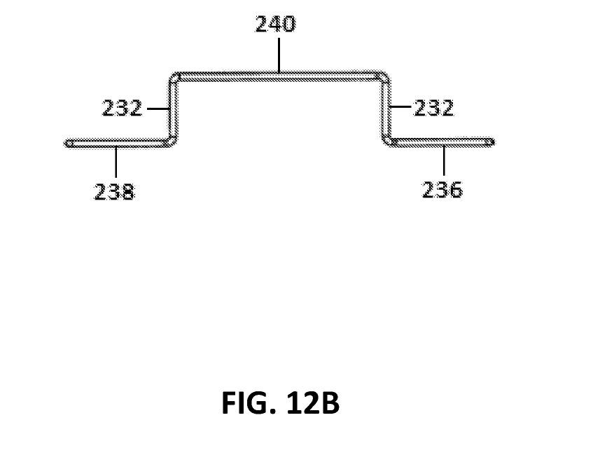

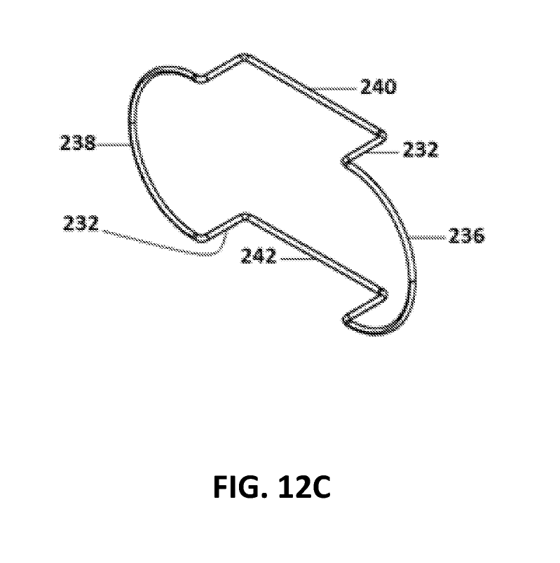

An alternative embodiment with a non-coplanar inner ring is shown in FIG. 11. In this embodiment, sheath 231 is joined to outer ring 202 and inner ring 234, which has a curved first portion 236 and an opposed curved second portion 238, separated on either end by rectangular portions 240, 242, which are displaced from the curved portions by step portions 232. The inner ring 234 is shown more closely in FIG. 12.

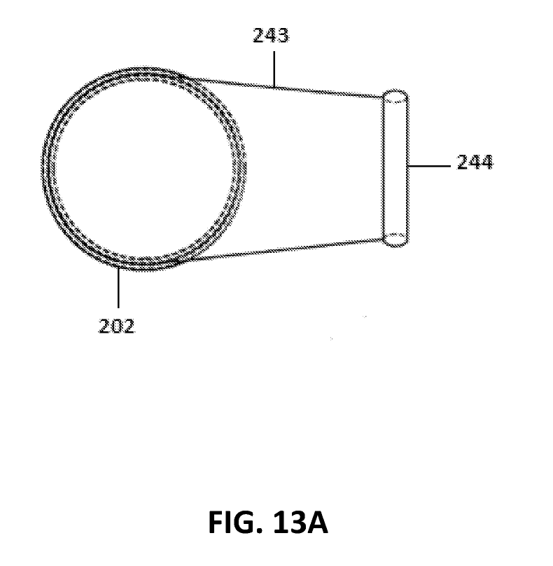

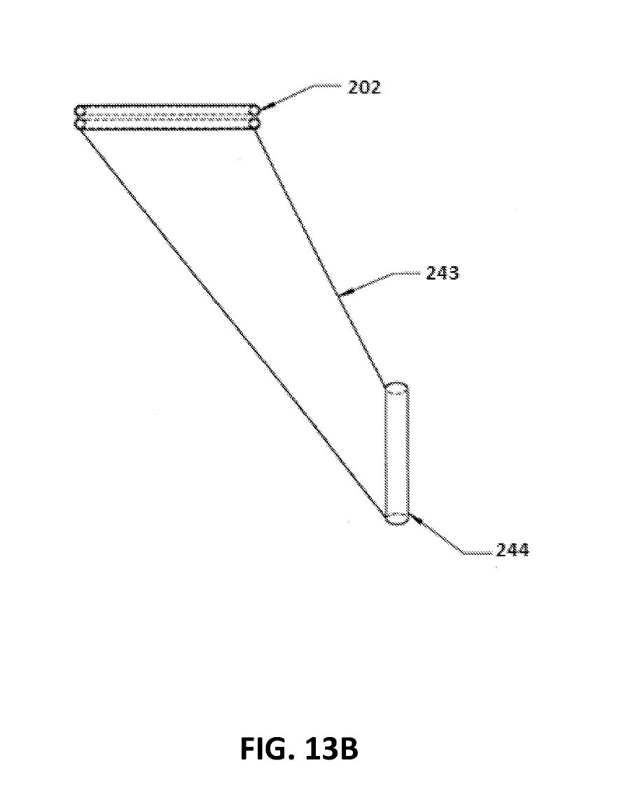

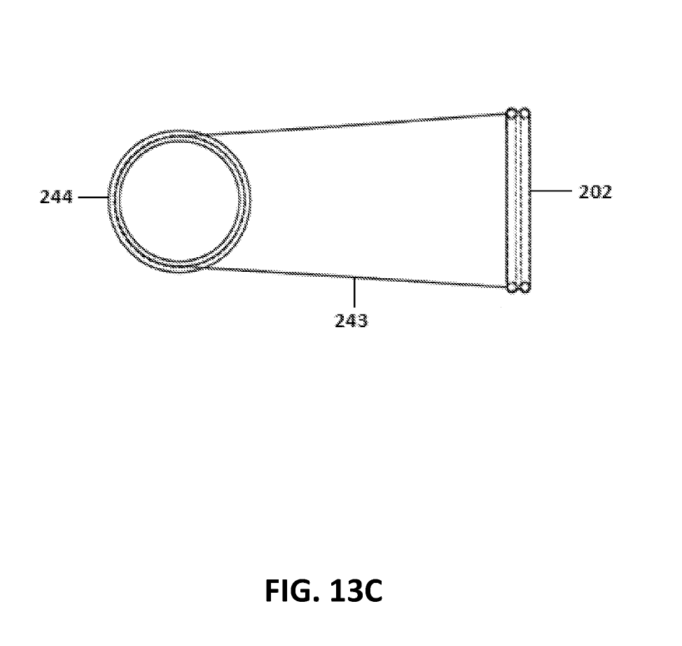

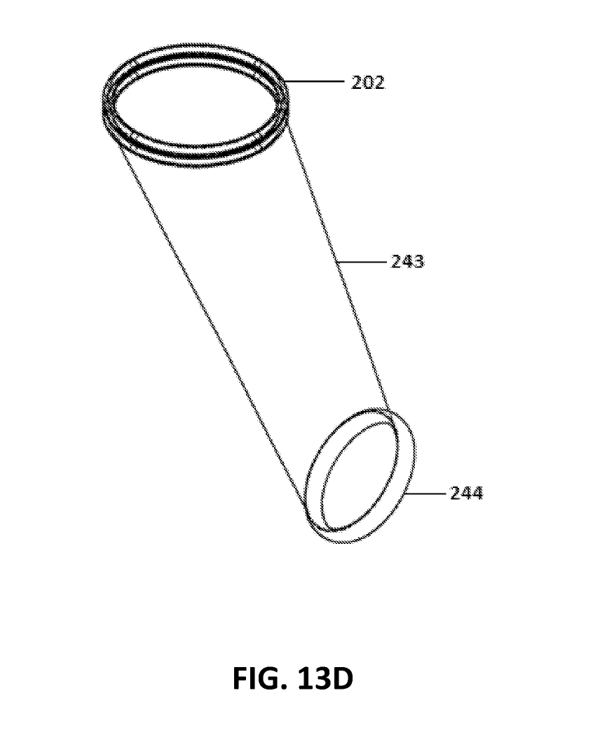

Alternatively, the inner ring can be configured to be non-parallel to the outer ring, one example of which is shown in FIG. 13. In FIG. 13, the sheath 243 is attached at either end to outer ring 202 and inner ring 244. The inner ring 244 is circular but is attached to the sheath 204 so as to form an angle, in this case, a 90-degree angle, with the outer ring 202. The inner ring can be disposed at a variety of angles relative to the outer ring, depending on the intended use.

In the non-coplanar and/or non-parallel embodiments shown in FIGS. 9-13, the design allows the user to create a space or pocket by deploying a section of the inner ring underneath the fascia layer. This separates the fascia layer from the surrounding tissue, creating a space in between. This may be useful in procedures such as hernia repair, for example.

In all of the disclosed embodiments, the inner ring must be capable of deformation for insertion through an incision or orifice and restoration to its original configuration following deployment. The inner ring can be made from a malleable material such as a soft metal, wire strands, a single core, or other materials known in the art. In some embodiments, the inner ring comprises a flexible material, for example, a polymer for example, a flexible engineering plastic. In some embodiments, the polymer is an elastomer, for example, a thermoplastic elastomer. In some embodiments, the inner ring is reshapeable, for example, comprising a plastically deformable or malleable elements, for example, metal and/or shape memory wires, strips, mesh, and the like.

In the illustrated embodiments, a cross section of the inner ring is generally circular or elliptical. However, the skilled artisan will appreciate that the inner ring may have another cross section, for example, oval, elliptical, flat, D-shaped, or any profile as described for the outer ring. The cross section of some embodiments of the inner ring is thinned and/or flattened at least at the outer edge, for example, a flat or thin wedge, resulting in an inner ring with a washer-like shape. The flattened outer edge permits a user to manipulate the edge into tight spaces when placing the inner ring, for example, between muscle layers. Embodiments of the inner ring are molded and/or extruded as a single piece, or as a plurality of pieces that are assembled into the inner ring.

It should be appreciated that the shape and position of the inner ring relative to the outer ring poses a unique challenge when compared to the standard retractor/protector of FIG. 1. In a retractor having a generally cylindrical sheath, as shown in FIG. 1, the diameter of the sheath along its length is generally constant, matching the diameters of the inner and outer ring. In such uses, a sheath material that is isotropic non-distensible may be preferred for maximum retraction.

In the embodiments described herein, however, the sheath may not be not cylindrical, but rather, for example, frustoconical, hourglass-shaped, D-shaped, oval, combinations, and the like as determined by the shape and size of the outer and inner ring.

In some embodiments, the sheath is fabricated as a seamless tube. In other embodiments, the sheath comprises at least one seam. In some embodiments, the sheath comprises longitudinal pleats.

Embodiments of the sheath comprise sheets, membranes, fibers, and/or strands of one or more materials that endow the sheath with the abrasion and puncture resistance. Suitable sheets, membranes, fibers, and/or strands comprise at least one of natural polymers, semi-synthetic polymers, synthetic polymers, metal, ceramic, glass, carbon fiber, carbon nanotubes, and the like. Suitable natural polymers include cellulose, silk, and the like. Semi-synthetic fibers include nitrocellulose, cellulose acetate, rayon, and the like. Suitable synthetic fibers include polyester, aromatic polyester, polyamide (NYLON®, DACRON®), aramid (KEVLAR®), polyimide, polyolefin, polyethylene (SPECTRA®), polyurethane, polyurea, polyvinyl chloride (PVC), polyvinylidene chloride, polyether amide (PEBAX®), polyether urethane (PELLETHANE®), polyacrylate, polyacrylonitrile, acrylic, polyphenylene sulfide (PPS), polylactic acid (PLA), poly(diimidazopyridinylene-dihydroxyphenylene) (M-5); poly(p-phenylene-2,6-benzobisoxazole) (ZYLON®), liquid crystal polymer fiber (VECTRAN®), and the like, and blends, copolymers, composites, and mixtures thereof. Suitable metals include stainless steel, spring steel, nitinol, super elastic materials, amorphous metal alloys, and the like.

Some embodiments of the sheath material comprises a composite comprising a fabric or textile, for example, at least one of a coated fabric, a laminated fabric, and a fabric embedded in a polymer. Coatings and/or laminations are disposed on one face or both faces of the fabric. Suitable coatings and laminating materials include polymers, for example, at least one of polyurethane, polyether, PVC, polyvinylidene chloride, silicone, styrene-butadiene, polyethylene, polypropylene, ethylene-propylene copolymer, polyisoprene, ethylene vinyl acetate (EVA), ethylene-propylene-diene monomer (EPDM), polyamide (MYLAR®), polyether block amide (PEBAX®), polyether urethane (PELLETHANE®), composites, blends, mixtures, and the like. An example of a suitable composite fabric is polyurethane laminated fabric (PUL). Some embodiments of the coating or lamination modify gas and/or moisture permeability through the sheath material, for example, by controlling the size of pores therethrough. For example, decreasing moisture permeability reduces dehydration of the retracted tissue and/or creates a barrier to pathogens such as bacteria. Increasing gas and moisture permeability permits hydrating and/or oxygenating the retracted tissue. Some materials are selectively permeable to certain fluids. For example, some embodiments of PVC are oxygen permeable and moisture impermeable, thereby permitting simultaneously oxygenating tissue while reducing dehydration. Some embodiments of the coating or lamination comprise an antibacterial or antimicrobial agent. In some embodiments, the antibacterial or antimicrobial agent is a surface agent or is integral to the material. Examples of suitable antibacterial or antimicrobial agents include iodine, antibiotics, silver, triclosan, biocides, and the like. Some embodiments of the coating or lamination provide a smoother and/or lower friction inside surface, which reduces the likelihood of instrument damage to the sheath.

Some embodiments of the sheath comprise a composite comprising a fiber-reinforced polymer film or membrane. Suitable fibers or strands are discussed above. Suitable polymer film materials include at least one of materials discussed above as coating and laminating materials. In some embodiments, the fibers are sandwiched between polymer film layers. In some embodiments, the polymer film layers are independently selected. For example, in some embodiments, the outer layer provides desirable tissue contact properties discussed above, while the inner layer is puncture resistant.

Some embodiments of the sheath comprise a plurality of layers, for example, a fabric layer and a polymer film layer, or a fabric layer sandwiched between polymer film layers. In some embodiments, the layers are secured to each other. In other embodiments, the layers are independent of, or not secured to each other, for example, a polymer film layer and a layer comprising a plurality of strips or bands as discussed above.

Some embodiments of the sheath comprise a fluid-permeable layer disposed on a fluid-impermeable layer, with the fluid-impermeable layer disposed on the inside of the sheath. The fluid-permeable layer contacts the wound margins, thereby permitting a user to supply pressurized fluid and/or apply vacuum to the wound margins. For example, in some embodiments, oxygen, moisture, therapeutic agent, and/or other fluids are supplied to the wound margins. In some embodiments, applying vacuum promotes bleeding, thereby reducing tissue necrosis. Embodiments of the fluid-permeable layer comprise at least one of open cell foam, fabrics, non-woven fabrics, and knit fabrics.

In other embodiments, the sheath is stretchable longitudinally. In some embodiments, longitudinal and circumferential stretch characteristics of the sheath are the same, that is, the stretch is isotropic.

In other embodiments, longitudinal and circumferential stretch characteristics of the sheath are different, that is, the stretch is anisotropic. For example, in some embodiments, the sheath has greater circumferential stretch than longitudinal stretch. A sheath having anisotropic stretch characteristics is particularly important in retractor/protectors having outer and inner rings of different sizes or shapes.

For example, for a retractor having a larger outer ring and a smaller inner ring, if the sheath diameter matches the diameter of a larger outer ring, the sheath material will bunch, pleat or fold at the smaller ring. Since the retractor is providing access to the surgical site, it is important to keep the working channel of the retractor open and clear of extraneous matter. Thus having a cylindrical sheath with a constant diameter equal to that of the larger outer ring is not preferable.

If the sheath diameter matches the diameter of smaller, inner ring, it must be elastic enough to circumferentially stretch to fit the larger outer ring. However, non-elastic sheaths provide better retraction. Good retraction is important to provide access to the surgical site, so having a cylindrical sheath with a constant diameter equal to that of the inner ring is also not preferable.

Using an anisotropic sheath material, circumferentially elastic to allow the sheath to attach to different size rings without bunching or folding but non-distensible in the longitudinal direction to provide better retraction, solves this problem. It is important that any such anisotropic material remains flexible, as retraction is achieved by rolling the sheath material around the outer ring after deployment.

In other embodiments, the sheath has substantially no or little longitudinal stretch, that is, is non-distensible longitudinally. Consequently, a retraction force exerted on an incision or opening by the sheath remains substantially constant over the course of a procedure. In some embodiments, the sheath is radially or circumferentially expandable. For example, some embodiments of a tubular sheath comprise a woven material that is expandable or stretchable circumferentially, that is, perpendicular to the longitudinal axis. Some embodiments comprise an elastomeric membrane or film, and longitudinal non-stretchable elements. For example, some embodiments of the sheath comprise a composite comprising an elastomeric film and longitudinally disposed, non-stretchable fibers, as discussed above. The fibers make the sheath longitudinally non-stretchable, while the polymer film permits radial expansion. Embodiments of the sheath comprising non-stretchable longitudinal strips and an elastomeric membrane are also longitudinally non-stretchable and radially expandable. Embodiments of a sheath comprising a non-stretchable tube comprising one or more longitudinal slits and/or pleats are longitudinally non-stretchable and radially expandable. Embodiments of a sheath comprising a plurality of non-stretchable longitudinal strips or bands are also longitudinally non-stretchable and radially expandable.

In some embodiments, at least a portion of the sheath is transparent, thereby providing a view of the retracted tissue. In some embodiments comprising a polymer membrane or film, the polymer membrane or film is transparent.

While certain embodiments have been particularly shown and described with reference to exemplary embodiments thereof, it will be understood by those of ordinary skill in the art that various changes in form and details may be made therein without departing from the spirit and scope thereof as defined by the following claims.

1. A retractor/protector comprising:
a longitudinal axis defining an instrument access channel extending from a proximal end to a distal end;
an outer ring;
an inner ring; and
a flexible sheath extending between the outer ring and the inner ring,
wherein
the instrument access channel extends through the outer ring, the inner ring, and the sheath, and
the inner ring is non-circular.

2. The retractor/protector of claim 1, wherein the inner ring is elliptical.

3. The retractor/protector of claim 1, wherein the inner ring comprises a plurality of spokes.

4. The retractor/protector of claim 1, wherein the inner ring is generally semi-circular.

5. The retractor/protector of claim 1, wherein the inner ring comprises a pair of spokes.

6. The retractor/protector of claim 1, wherein the inner ring comprises at least two pairs of spokes.

7. The retractor/protector of claim 1, wherein the outer ring is non-circular.

8. A retractor/protector comprising:
a longitudinal axis defining an instrument access channel extending from a proximal end to a distal end;
an outer ring;
an inner ring; and
a flexible sheath extending between the outer ring and the inner ring,
wherein
the instrument access channel extends through the outer ring, the inner ring, and the sheath, and
the inner ring is non-coplanar.

9. The retractor/protector of claim 8, wherein the inner ring comprises a first portion, a second portion, and a pair of step portions, the first portion and second portion being connected to each other by the pair of step portions.

10. The retractor/protector of claim 8, wherein the inner ring comprises a curved first portion, an opposed curved second portion, a rectangular first portion, an opposed rectangular second portion, and two pairs of step portions, each curved portion being connected on either end to a rectangular portion by a pair of step portions.

11. A retractor/protector comprising:
a longitudinal axis defining an instrument access channel extending from a proximal end to a distal end;
an outer ring;
an inner ring; and
a flexible sheath extending between the outer ring and the inner ring,
wherein
the instrument access channel extends through the outer ring, the inner ring, and the sheath, and
the inner ring is non-parallel to the outer ring.

12. The retractor/protector of claim 11, wherein the inner ring is attached to the sheath at a ninety degree angle to the outer ring.
